(19) United States
(12) Patent Application Publication
(10) Pub. No.: US 2017/0231952 A1
(43) Pub. Date: Aug. 17, 2017

(54) PHARMACEUTICAL AGENT

(71) Applicant: Skintech Life Science Limited, Stanmore, Middlesex (GB)

(72) Inventor: David ALPERT, London (GB)

(21) Appl. No.: 15/328,457

(22) PCT Filed: Jul. 22, 2015
(86) PCT No.: PCT/EP2015/066805

§ 371 Date: Jan. 23, 2017

(30) Foreign Application Priority Data
Jul. 23, 2014 (GB) 1413058.7

(51) Int. Cl. A61K 31/404; A61K 45/06; A61K 31/23

(52) U.S. Cl. CPC A61K 31/404; A61K 31/23; A61K 45/06

(57) ABSTRACT

Provided herein is a composition for use in treating rosacea, the composition comprising: (a) a first component comprising a substituted or unsubstituted diindolylmethane; and (b) optionally a second component comprising a substituted or unsubstituted retinoid compound.

### CROSS-REFERENCE

This application claims the benefit of Application No. GB1413058.7, filed Jul. 23, 2014, which is incorporated by reference herein in its entirety.

### BACKGROUND OF THE DISCLOSURE

Rosacea is a chronic skin condition characterized by facial redness, small and superficial dilated blood vessels on facial skin, papules, pustules, and swelling. Rosacea affects all ages.

### SUMMARY OF THE DISCLOSURE

Provided herein, in some embodiments, is a composition for use in treating rosacea, the composition comprising, (a) a first component comprising a substituted or unsubstituted diindolylmethane; and (b) optionally, a second component comprising a substituted or unsubstituted retinoid compound. In some embodiments, the composition for use in treating rosacea further comprises a further component selected from one or more of, a substituted or unsubstituted retinoid compound, an antibiotic compound, a substituted or unsubstituted azelaic acid compound, an oral contraceptive compound, sulphur, a sulphur-containing compound, a substituted or unsubstituted salicylic acid compound, a substituted or unsubstituted resorcinol compound, a plant product, a mineral, a vitamin, and a neutraceutical product. In some embodiments, the composition for use in treating rosacea is a diindolylmethane of Formula 1:

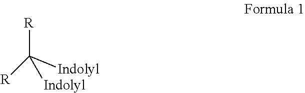

wherein the R groups are independently selected from hydrogen atoms and C₁-C₆ hydrocarbon substituents; and wherein the indolyl groups are independently selected from indole-3-yl and indole-2-yl groups; and wherein the indolyl groups are unsubstituted, or are substituted with one or more C₁-C₆ hydrocarbon substituents.

In some embodiments, the first component is selected from a group consisting of a substituted or unsubstituted 3,3′diindolylmethane, of Formula 2, and a substituted or unsubstituted 2,2′ diindolylmethane, of Formula 3:

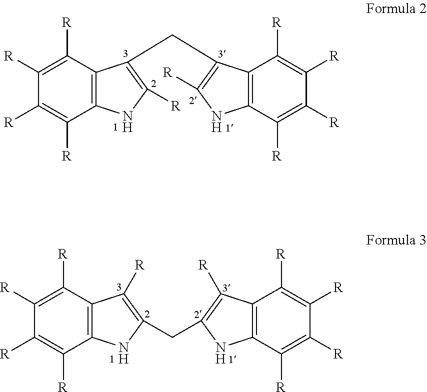

wherein the R groups are independently selected from hydrogen atoms, and C₁-C₆ hydrocarbon substituents.

In some embodiments, the first component is a 3,3′diindolylmethane, of Formula 4 or a 2,2′diindolylmethane, of Formula 5:

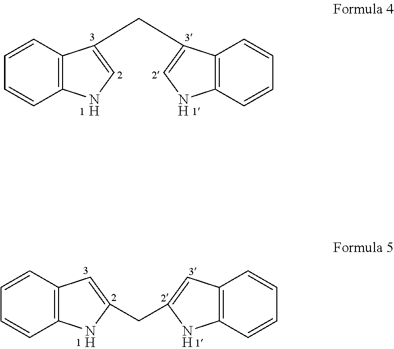 [SMILES: c1ccc2c(Cc3c[nH]c4ccccc34)c[nH]c2c1 ; c1ccc2[nH]c(Cc3cc4ccccc4[nH]3)cc2c1]

In some embodiments, the substituted or unsubstituted retinoid compound is selected from a substituted or unsubstituted first generation retinoid, a substituted or unsubstituted second generation retinoid, and a substituted or unsubstituted third generation retinoid. In some embodiments, the substituted or unsubstituted first generation retinoid is selected from a substituted or unsubstituted retinol, a substituted or unsubstituted retinal, a substituted or unsubstituted tretinoin, a substituted or unsubstituted isoretinoin, and a substituted or unsubstituted alitretinoin, wherein the substituted or unsubstituted tretinoin is selected from retinoic acid and Retin A. In some embodiments, the first generation retinoid is vitamin A. In some embodiments, the second generation retinoid is selected from a substituted or unsubstituted etretinate, and a substituted or unsubstituted acitretin. In some embodiments, the third generation retinoid is selected from a substituted or unsubstituted tazarotene, a substituted or unsubstituted bexarotene, and a substituted or unsubstituted adapalene.

In some embodiments, the antibiotic compound is selected from tetracycline, oxytetracycline, minocycline, doxycycline, erythromycin, and trimethoprim. In some embodiments, the azelaic acid compound comprises an antibacterial property, an anti-inflammatory property, or a combination thereof. In some embodiments, the oral contraceptive comprises a female hormone, a compound with female hormone activity, a compound with female hormone activity capable of counteracting the effect of a male hormone, or a combination thereof. In some embodiments, the female hormone is oestrogen and the male hormone is testosterone. In some embodiments, the resorcinol compound comprises an antibacterial property, an anti-inflammatory property, or a combination thereof.

In some embodiments, the composition for use in treating rosacea, comprises a first component comprising a substituted or unsubstituted diindolylmethane, and optionally a second component comprising a substituted or unsubstituted retinoid compound, wherein an unsubstituted compound comprises only hydrogen atom substituents, and wherein a substituted compound comprises one or more C1-C6 hydrocarbon substituents.

In some embodiments, the type of rosacea is selected from Pustular rosacea, Papulopustular rosacea, Erythemato-telangiectatic rosacea, Granulomatous rosacea, Rosacea conglobata, Rosacea fulminans, and Phymatous rosacea.

In some embodiments, the composition is suitable for oral or topical administration. In some embodiments, the composition is in the form of a tablet, capsule, gel, cream or ointment.

Provided herein in some embodiments, is a pharmaceutical kit comprising a first component and optionally a second component, for use in treating a skin condition, by separate, simultaneous or sequential administration of the first and the second component, wherein the first and second components comprise a substituted or unsubstituted diindolylmethane, and a substituted or unsubstituted retinoid compound, respectively. In some embodiments, the substituted or unsubstituted diindolylmethane is synthetic, or is a natural product obtained from a Brassica plant. In some embodiments, the substituted or unsubstituted diindolylmethane is adapted for increased bioavailability. In some embodiments, the substituted or unsubstituted diindolylmethane is BR-DIM.

In some embodiments, the kit comprises the first component, the second component, the further component, an excipient or adjuvant, and a combination thereof. In some embodiments, the first component further comprises one or more lipophilic compounds selected from vitamin E and phosphatidylcholine. In some embodiments, the first component is in a form suitable for providing a daily substituted or unsubstituted diindolylmethane dosage of up to 1500 mg or less. In some embodiments, the first component is in a form suitable for providing a daily substituted or unsubstituted diindolylmethane dosage of about 10 mg to about 750 mg. In some embodiment, the first component is for treating rosacea, and is in a form suitable for providing a daily substituted or unsubstituted diindolylmethane dosage of about 150 mg to about 650 mg, about 250 mg to about 550 mg, about 300 mg to about 500 mg, about 400 mg to about 450 mg, or about 500 mg. In some embodiment, the first component is present in a dose which is a fraction of the daily dose, preferably a half of the daily dose, or a quarter of the daily dose. In some embodiments, second component is in a form suitable for providing a daily retinoid compound dosage of about 200 to about 3000 μg.

In some embodiments, the daily dosage is provided in the form of one or more capsules or tablets.

Provided herein, in some embodiments, is a method for treating rosacea, comprising administering a composition or components of a kit, as described herein, to a subject in need thereof. Provided herein, in some embodiments, is a method for preparing a composition for use in treating rosacea, as described herein, comprising mixing the first component with one or more of the second component, the excipients, and the additives, to form the composition.

In some embodiments, the first and second components are each, separately from each other, mixed with one or more excipients, additives, or a combination thereof, before being mixed together to form the composition.

Provided herein, in one embodiment, is a substituted or unsubstituted diindolylmethane for use in treating rosacea.

### BRIEF DESCRIPTION OF THE FIGURES

FIG. 1 shows patient A1 with rosacea prior to (top) and after (bottom) treatment with an exemplary BR-DIM composition, 4 capsules per day for 6 months.

FIG. 2 shows patient A2 with rosacea prior to (top) and after (bottom) treatment with an exemplary BR-DIM composition, 4 capsules per day for 3 months.

FIG. 3 shows patient A3 with rosacea prior to (top) and after (bottom) treatment with an exemplary BR-DIM composition, 2 capsules per day for 11 weeks.

FIG. 4 shows patient B1 with rosacea prior to (top) and after (bottom) treatment with an exemplary BR-DIM composition, 4 capsules per day for 6 months.

FIG. 5 shows patient B2 with rosacea prior to (top) and after (bottom) treatment with an exemplary composition, for 3 months.

FIG. 6 shows a VISIA redness picture of patient A1 with rosacea prior to (top) and after (bottom) treatment with an exemplary BR-DIM composition, for 6 months.

FIG. 7 shows a VISIA redness picture of patient A2 with rosacea prior to (top) and after (bottom) treatment with an exemplary BR-DIM composition, for 3 months.

FIG. 8 shows a VISIA redness picture of patient B1 with rosacea prior to (top) and after (bottom) treatment with an exemplary BR-DIM composition, for 6 months.

FIG. 9 shows a VISIA redness picture of patient B2 with rosacea prior to (top) and after (bottom) treatment with an exemplary BR-DIM composition, for 3 months.

### DETAILED DESCRIPTION OF THE DISCLOSURE

The present application discloses a pharmaceutical composition or kit comprising a substituted or unsubstituted diindolylmethane (DIM) for use in treating rosacea. In some embodiments, the composition optionally comprise one or more other components in addition to the substituted or unsubstituted DIM in order to enhance the pharmaceutical effect of the substituted or unsubstituted DIM.

In general, rosacea has not been widely investigated, especially compared to acne and other skin diseases. In the present disclosure, it is unexpectedly discovered that DIM alone, or with certain further beneficial additives, is effective for improved treatment and prevention of rosacea.

As such, provided herein, in one embodiment, are methods and compositions for use in treating rosacea, wherein the composition comprises a first component, optionally a second component, wherein the first component comprises a substituted or unsubstituted diindolylmethane (DIM), and the second component comprises a substituted or unsubstituted retinoid compound. In some embodiments, are provided kits containing the compositions for use in treating rosacea, as described herein. In some embodiments, the DIM is a substituted or an unsubstituted DIM. In some embodiments, the retinoid compound is a substituted or an unsubstituted retinoid compound. In some embodiments, the DIM is BioResponse DIM (BR-DIM). In some embodiments, the DIM is any compound comprising a diindolylmethane group. In some embodiments, the DIM has the following structure (Formula 1):

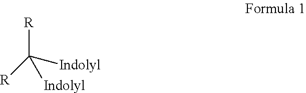

wherein the R groups are be the same or different substituents. In some embodiments, the R substituents comprise any organic group and/or one or more atoms from any of groups IIIA, IVA, VA, VIA or VIIA of the Periodic Table, such as a B, Si, N, P, O, or S atom or a halogen atom (e.g., F, Cl, Br or I).

In some embodiments, the R substituent comprises an organic group. In some embodiments, the organic group comprises a hydrocarbon group. In some embodiments, the hydrocarbon group comprises a straight chain, a branched chain or a cyclic group. In some additional embodiments, the hydrocarbon group comprises an aliphatic or an aromatic group. In some additional embodiments, the hydrocarbon group comprises a saturated or an unsaturated group.

In some embodiments, the hydrocarbon comprises an unsaturated group comprising one or more alkene functionalities and/or one or more alkyne functionalities. In some embodiments, the hydrocarbon comprises a straight or branched chain group comprising one or more primary, secondary and/or tertiary alkyl groups. In some embodiment, the number of carbon atoms in the hydrocarbon group is between 1-40. In some embodiments, the hydrocarbon group is a lower hydrocarbon. In some embodiments, the number of carbon atom in the lower hydrocarbon is between 1 and 6. In some additional embodiments, the hydrocarbon group is a higher hydrocarbon. In some embodiments, the number of hydrocarbons in the higher hydrocarbon is higher than 7. In some embodiments, the number of carbon atoms in the higher hydrocarbon is between 7 and 40.

In some embodiments, the hydrocarbon comprises a cyclic group comprising an aromatic ring, an aliphatic ring, a heterocyclic group, and/or fused ring derivatives of these groups. In some embodiments, the cyclic group comprises a benzene, a naphthalene, an anthracene, an indene, a fluorene, a pyridine, a quinoline, a thiophene, a benzothiophene, a furan, a benzofuran, a pyrrole, an indole, an imidazole, a thiazole, and/or an oxazole group, as well as regioisomers of the above groups. In some embodiments, the number of atoms in the ring of the cyclic group is between 3 and 10. In some embodiments, the number of atoms in the ring of the cyclic group is 3, 4, 5, 6, or 7.

In some embodiments, the cyclic groups comprising heteroatoms described above, as well as any of the other groups defined above, further comprise one or more heteroatoms from any of groups IIIA, IVA, VA, VIA or VIIA of the Periodic Table, such as a B, Si, N, P, O, or S atom or a halogen atom (e.g. F, Cl, Br or I).

In some embodiments, the R substituent comprises one or more of any of the common functional groups in organic chemistry, such as hydroxy groups, carboxylic acid groups, ester groups, ether groups, aldehyde groups, ketone groups, amine groups, amide groups, imine groups, thiol groups, thioether groups, sulphate groups, sulphonic acid groups, and phosphate groups etc. In some embodiments, the substituent comprises one or more derivatives of any of the common functional groups in organic chemistry, such as hydroxy groups, carboxylic acid groups, ester groups, ether groups, aldehyde groups, ketone groups, amine groups, amide groups, imine groups, thiol groups, thioether groups, sulphate groups, sulphonic acid groups, and phosphate groups etc. Exemplary derivatives include but are not limited to, carboxylic acid anhydrydes and carboxylic acid halides.

In some embodiments, any R substituent comprises a combination of two or more of the substituents and/or functional groups defined above.

In some embodiments, the R substituents are selected from hydrogen atoms and C₁-C₆ hydrocarbon substituents such as C₁-C₆ alkyl groups (e.g, methyl, ethyl, propyl, isopropyl, and butyl groups). In some embodiments, both R substituents are hydrogen atoms.

In some embodiments, the indolyl groups in formula 1 are the same or different. In some embodiments, both indolyl groups are indole-3-yl groups. In some embodiments, both indolyl groups are indole-2-yl groups. In some embodiments, one indolyl group is indole-3-yl and the other indolyl group is indole-2-yl. In some embodiments, the indolyl groups comprise same or different substituents. In some embodiments, the indolyl groups are unsubstituted, such that all substituents are hydrogen atoms. In some embodiments, the indolyl groups are substituted with one or more of any of the R substituents defined above. In some embodiments, the substituents are selected from hydrogen atoms and C₁-C₆ hydrocarbon substituents such as C₁-C₆ alkyl groups (e.g., methyl, ethyl, propyl, isopropyl, and butyl groups).

In some embodiments, the composition comprises a first component comprising a substituted or unsubstituted 3,3′diindolylmethane, of Formula 2. In some embodiments, the composition comprises a first component comprising a substituted or unsubstituted 2,2′ diindolylmethane, of Formula 3.

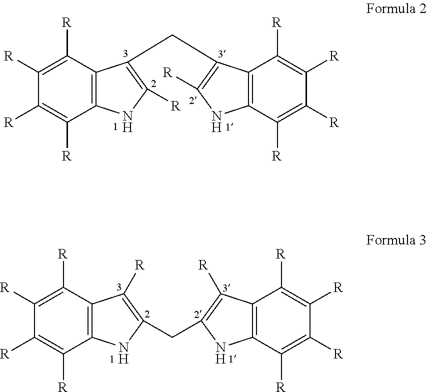

wherein the R groups are independently selected from hydrogen atoms, or C₁-C₆ hydrocarbon substituents.

In some embodiments, the composition comprises a first component comprising a 3,3′diindolylmethane, of Formula 4. In some embodiments, the composition comprises a first component comprising a 2,2′diindolylmethane, of Formula 5.

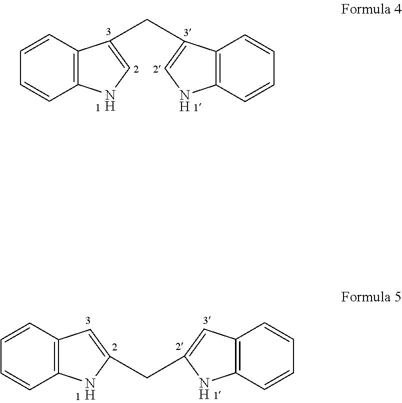 [SMILES: c1ccc2c(Cc3c[nH]c4ccccc34)c[nH]c2c1 ; c1ccc2[nH]c(Cc3cc4ccccc4[nH]3)cc2c1]

In some embodiments, the compositions and kits described herein, comprise a second component comprising a substituted or unsubstituted retinoid compound. The second component typically has a beneficial effect supplementary to and/or complementary to the effect of the first component. In some embodiments, the effect of the second component is complementary to that of the first component. In some embodiments, when the effect of the second component differs from that of the first component, but is additionally beneficial for treatment of rosacea, the second component is said to have an effect that is complementary to the first component. In some embodiments, the effect of the second component is supplementary to that of the first component. In some embodiments, when the effect of the second component is same as that of the first component, and further produces an additive effect, or enhances the pharmaceutical effect of the first component, the second component is said to have an effect that is supplementary to that of the first component. In some embodiments, the first component is a DIM. In some embodiment, the second component is any component that has a synergistic effect or any other beneficial relationship with the first component. In some embodiment, the first component is a DIM or a BR-DIM and the second component is a retinoid compound. In some embodiment, the first component is a DIM of Formula 1 and the second component is a retinoid compound. In some embodiment, the first component is a DIM of Formula 1 and the second component is vitamin A. In some embodiment, the first component is a BR-DIM and the second component is a retinoid compound. In some embodiment, the first component is a BR-DIM and the second component is vitamin A.

In some embodiments, the retinoid compound of the second component is any such compound known in the art that is suitable for use with the skin. For example, in some embodiments, it is selected from a substituted or unsubstituted first generation retinoid, a substituted or unsubstituted second generation retinoid, and a substituted or unsubstituted third generation retinoid. In some embodiments, the retinoid is a substituted or unsubstituted first generation retinoid. In some embodiments, the substituted or unsubstituted first generation retinoid is selected from a substituted or unsubstituted retinol, a substituted or unsubstituted retinal, a substituted or unsubstituted tretinoin (e.g., retinoic acid or Retin A), a substituted or unsubstituted isotretinoin (e.g, Accutane™), and a substituted or unsubstituted alitretinoin. In some embodiments, the retinoid is vitamin A. In some embodiments, the retinoid is a substituted or unsubstituted second generation retinoid selected from a substituted or unsubstituted etretinate, and a substituted or unsubstituted acitretin. In some embodiments, the retinoid is a substituted or unsubstituted third generation retinoid selected from a substituted or unsubstituted tazarotene, a substituted or unsubstituted bexarotene, and a substituted or unsubstituted adapalene.

In one embodiment, is provided a composition, wherein the first component is an unsubstituted 3,3′diindolylmethane and the second component is a vitamin A compound (e.g, vitamin A palmitate).

In one embodiment, is provided a kit, containing the compositions described herein. In one embodiment, is provided a kit containing a composition consisting of a first component and a second component, wherein the first component is an unsubstituted 3,3′diindolylmethane and the second component is a vitamin A compound (e.g, vitamin A palmitate).

As such provided, in another embodiment described herein, is a composition for use in treating rosacea, the composition comprising:

(a) a first component comprising a substituted or unsubstituted diindolylmethane; and
(b) optionally a second component different from the first component, wherein the second component has a beneficial effect supplementary to and/or complementary to the effect of the first component, wherein the second component is selected from an antibiotic compound, a substituted or unsubstituted azelaic acid compound, an oral contraceptive compound, sulphur, a sulphur-containing compound, a substituted or unsubstituted salicylic acid compound, a substituted or unsubstituted resorcinol compound, a plant product, a mineral, a vitamin, and a neutraceutical product, and wherein the second component is not a retinoid compound.

In some embodiments, the composition optionally comprises one or more further components. In some embodiments, the one or more further components are present when the second component is absent. In some other embodiments the one or more further components are present when the second component is also present. In some embodiments, the one or more further components are beneficial for the treatment of rosacea. In some embodiments, the one or more further components, provide some beneficial effect not related to the treatment of rosacea, but beneficial to skin generally. In some embodiments, the one or more further components are selected from a further substituted or unsubstituted retinoid compound, an antibiotic compound, a substituted or unsubstituted azelaic acid compound, an oral contraceptive compound, sulphur, a sulphur-containing compound, a substituted or unsubstituted salicylic acid compound, a substituted or unsubstituted resorcinol compound, a plant product, a mineral, a vitamin (typically not vitamin A which instead in some embodiments is present as a retinoid compound), and a neutraceutical product. Thus, additional further components in some embodiments include one or more of: omega 3, omega 6, B vitamins including folic acid and vitamin B12, vitamin C, vitamin D, vitamin E, betaine hydrochloride, pepsin, one or more probiotics, zinc, kelp, one or more minerals, one or more herbal supplements, one or more polyphenolic compounds from the bergamot fruit (Citrus bergamia) preferably when extracted and concentrated, and bergamot.

In some embodiments, the further component comprises either one or more of the further components described above, depending upon the requirements of the formulation.

In some embodiments, the antibiotic compound is any such compound known in the art that is suitable for use with the skin. In some embodiments, the antibiotic compound is suitable for oral and/or topical application and is selected from tetracycline, oxytetracycline, minocycline, doxycycline, erythromycin and trimethoprim.

In some embodiments, the azelaic acid compound is any such compound known in the art that is suitable for use with the skin. In some embodiments, the azelaic acid compound suitable for use with the skin comprises an antibacterial and/or an anti-inflammatory property.

In some embodiments, the oral contraceptive compound is any such compound known in the art that is suitable for use with the skin. In some embodiments, the oral contraceptive comprises a female hormone and/or a compound with female hormone activity, capable of counteracting the effect of a male hormone. In some embodiments, the female hormone is estrogen and the male hormone is testosterone.

In some embodiments, the sulphur or sulphur-containing compound is any such compound known in the art that is suitable for use with the skin. In some embodiments, the sulphur or sulphur-containing compound comprises sulphur either alone or in combination with other substances such as alcohol, salicylic acid and/or resorcinol.

In some embodiments, the resorcinol compound is any such compound known in the art that is suitable for use with the skin. In some embodiments, the resorcinol compound comprises a compound with an antibacterial and/or an anti-inflammatory property.

In some embodiments, the plant product is an extract from one or more of the following:
Urtica dioica (stinging nettle)—has significant anti-inflammatory effects helping to reduce the redness and swelling in acne
Ribes nigrum (blackcurrant)—has significant anti-inflammatory and antioxidant properties
Echinacea purpurea and/or Echinacea angustifolia—contain active substances that enhance the activity of the immune system, reduce inflammation and antioxidant effects
Cnidium monnieri—acts through its antibacterial, antifungal, and astringent properties
Berberis aquafolium (Oregon grape)—in some embodiments decrease sebum production, and reduce infection and inflammation
Chamomile Matricaria (German chamomile)—has antibacterial properties and inactivates bacterial toxins. In some embodiments reduce inflammation, and speed wound healing
Phytolacca decandra (poke root)
Cruciferous vegetables—synergistic effects in some embodiments are achieved by the addition of extracts, concentrates and processed extracts of cruciferous vegetables such as broccoli, sprouts etc. These in some embodiments provide active products with significant added antioxidant and anti-inflammatory effects.

In some embodiments, the mineral comprises one or more of:
Zinc—in some embodiments promote reduction of inflammation and healing of damaged skin
Chromium—in some embodiments help boost the body's ability to break down glucose
Selenium—in some embodiments help to balance decreased levels of antioxidant enzymes and/or in some embodiments reduce persistent acne and help lessen the appearance of scars.

In some embodiments, the vitamin comprises one or more of:
Vitamin B complex—combinations of B vitamins B1, B2, B3, B4, B5, B6, B7, B9 and B12—in some embodiments balance the metabolism of hormones, which have a significant role in the condition of rosacea
Vitamin C—a highly effective antioxidant, which stimulates tissue growth and repair. In some embodiments contribute to the rosacea healing process
Vitamin E—a potent antioxidant

In some embodiments, the neutraceutical product has some further nutritional or pharmaceutical property that is complementary or supplementary to the effect of the first and/or second component. In some embodiments, one or more neutraceutical products are selected from:
Evening primrose oil—contains fatty acids, which in some embodiments help decrease inflammation related to rosacea
Plant isoflavones—from foods including soy bean and red clover—have significant natural antioxidant properties and a balancing effect on estrogenic hormones
Turmeric—has anti-inflammatory properties from the primary active component curcumin
Essential and non-essential fatty acids, such as omega fatty acids (omega-3, omega-6, etc.—long-chain n-3 fatty acids in some embodiments have anti-inflammatory activity

In some embodiments, the compound of the second component is substituted or unsubstituted. In some embodiments, the compound of the further component is substituted or unsubstituted. In some embodiments, the compounds of the second component and the further component are substituted or unsubstituted. In some embodiments, the compounds are substituted with any of the substituents described above, for the compound of Formula 1. In some embodiments, the substituents are selected from hydrogen atoms and C₁-C₆ hydrocarbon substituents such as C₁-C₆ alkyl groups (e.g. methyl, ethyl, propyl, isopropyl, and butyl groups). In some additional embodiments, all substituents are hydrogen atoms, i.e. the compounds are unsubstituted.

In some embodiments, the compositions described herein, further include additive, adjuvants and/or excipient compounds. In some embodiments, these additives, excipients and adjuvants enhance the effectiveness of the first and/or second components, or are advantageous in forming a working formulation for the pharmaceutical. Exemplary excipients and additive compounds include one or more of vitamin E, vitamin C, phosphatidyl choline, microcrystalline cellulose (plant fibre), magnesium stearate and silica. In some embodiments, the first component comprises a DIM or a BR-DIM and one or more additive, adjuvants and/or excipients. Diindolylmethane is a lipophilic oil-soluble compound. Similar to other oil soluble phytochemicals, the presence of oil and other lipophilic compounds, such as phosphatidylcholine and vitamin E, greatly increases the absorption and bioavailability of diindolylmethane upon oral administration. Thus, in some embodiments, the first component comprises a DIM or a BR-DIM and one or more further oil-soluble and/or lipophilic compounds, such as vitamin E and/or phosphatidylcholine. In some embodiments, further additives are also added to the first and/or the second component. These include, but are not limited to, microcrystalline cellulose (plant fibre), magnesium silicate, magnesium stearate, tri-calcium phosphate, and silica.

In some embodiments, the compositions and kit components described herein, are particularly effective against rosacea. In some embodiments, the type of rosacea is selected from Pustular rosacea, Papulopustular rosacea, Erythemato-telangiectatic rosacea, Granulomatous rosacea, Rosacea conglobata, Rosacea fulminans, and Phymatous rosacea. In some embodiments, the compositions and kit components described herein, are effective of treatment of rosacea in a patient. In some embodiments, the patient is any human patient (male or female; child or adult). In some additional embodiments, the human subject is an older person, aged 40 and above, 50 and above, 60 and above, or 70 and above. In some embodiments, the human subject is a female aged 40 and above.

In some embodiments, the compositions and the kit components described herein are used to prepare a formulation suitable for administration to a human or other subject. In some embodiments, the composition and the kit components are formulated for oral or topical administration. In some embodiments, the composition and/or the kit components, are provided in the form of a tablet, capsule, liquid (such as in the form of a drink, e.g., a yoghurt drink), gel, cream, or ointment. In some embodiment, the composition and/or the kit components comprises the first component present in a first formulation, and the optional second component present in a second formulation, wherein the first and second formulations are co-administered to a patient, either concurrently or in a sequential manner. In some embodiments, the composition and/or the kit components comprises the first component and second component present in one formulation that is administered to a patient.

### Bioavailability of Substituted or Unsubstituted DIM

Diindolylmethane (DIM) is a natural compound formed during the autolytic breakdown of glucobrassicin present in food plants of the Brassica genus, including broccoli, cabbage, Brussels sprouts, cauliflower and kale. The autolytic breakdown of glucobrassicin requires the catalytic reaction of the enzyme myrosinase, which is endogenous to these plants and released upon rupture of the cell wall. The compound is normally manufactured by chemical synthesis but in some embodiments is also prepared by natural means from the extracts of Brassica vegetables, as listed above, particularly from sprouting broccoli or from broccoli seeds. Thus, the substituted or unsubstituted DIM in some embodiments is synthetic, or in some embodiments is a natural product obtained from a Brassica plant, as discussed above. In some embodiments, the composition described herein, includes as a first component, any substituted or unsubstituted DIM, or a combination of the substituted or unsubstituted DIMs described above.

In some embodiments, the substituted or unsubstituted DIM is adapted for increased bioavailability, in order to reduce the required dosage. In some embodiments, the DIM with increased bioavailability is an unsubstituted DIM. In some embodiments, the DIM with increased bioavailability is BR-DIM (from BioResponse LLC).

### Dosage of Administration

In some embodiments, the composition comprising a first component comprising a substituted or unsubstituted DIM, is administered at a dosage that is sufficiently low to avoid toxicity, whilst still maintaining the required pharmaceutical effect. In some embodiments, the dosage depends on the bioavailability of the substituted or unsubstituted DIM. In some embodiments, the dosage varies depending upon whether it is a natural or synthetic product. In some embodiments, the dosage of the first component comprising the substituted or unsubstituted DIM is determined by whether or not the DIM has been adapted to improve bioavailability.

In some embodiments, the first component comprising a substituted or unsubstituted DIM is in a form suitable for providing a daily DIM dosage of 1500 mg or less, 1000 mg or less, 900 mg or less, 800 mg or less, 700 mg or less, 600 mg or less, 500 mg or less, from 10-750 mg, from 150-650 mg, from 250-550 mg, and from 300-500 mg. In some embodiments, the first component comprising a substituted or unsubstituted DIM is in a form suitable for providing a daily DIM dosage of about 300 mg or about 350 mg, or about 400 mg or about 450 mg, or about 500 mg. In some embodiments, the first component comprising a BR-DIM is in a form suitable for providing a daily BR-DIM dosage of 1500 mg or less, 1000 mg or less, 900 mg or less, 800 mg or less, 700 mg or less, 600 mg or less, 500 mg or less, from 10-750 mg, from 150-650 mg, from 250-550 mg, and from 300-500 mg. In some embodiments, the first component comprising a BR-DIM is in a form suitable for providing a daily BR-DIM dosage of about 300 mg or about 350 mg, or about 400 mg or about 450 mg, or about 500 mg. In some embodiments, the first component comprising a BR-DIM that has not been adapted to improve bioavailability, is administered in higher dosages.

In some embodiments, the first component comprising a substituted or unsubstituted DIM is in a form suitable for providing an intermediate daily DIM dosage, such as from 50-350 mg, 100-200 mg, 140-160 mg, or about 150 mg, or alternatively 250-350 mg, 290-310 mg or about 300 mg. In some embodiments, the first component comprising a substituted or unsubstituted DIM is in a form suitable for providing a low daily DIM dosage, such as from 15-100 mg, 50-100 mg, 18-80 mg, 18-75 mg, 70-80 mg, or about 75 mg. In some embodiments, the first component comprising a BR-DIM is in a form suitable for providing an intermediate daily BR-DIM dosage, such as from 50-350 mg, 100-200 mg, 140-160 mg, or about 150 mg, or alternatively 250-350 mg, 290-310 mg or about 300 mg. In some embodiments, the first component comprising a BR-DIM is in a form suitable for providing a low daily BR-DIM dosage, such as from 15-100 mg, 50-100 mg, 18-80 mg, 18-75 mg, 70-80 mg, or about 75 mg.

In some embodiments, the first component comprising a substituted or unsubstituted DIM that has been adapted to improve bioavailability is administered in intermediate and lower dosages. In some embodiments, the first component comprising a substituted or unsubstituted DIM that has been adapted to improve bioavailability is administered in any of the above dosages, including the higher dosages, if desired. In some embodiments, the DIM that has been adapted to improve bioavailability is BR-DIM.

In some other embodiments, the first component comprising a substituted or unsubstituted DIM is present in a dose which is a fraction of the daily dose, such as a half of the daily dose, or a quarter of the daily dose, and thus is present in a half or a quarter of any of the dosages recited above. In these embodiments, each dose fraction is taken separately over time to spread the dose across the day. In some other embodiments, the first component comprising a BR-DIM is present in a dose which is a fraction of the daily dose, such as a half of the daily dose, or a quarter of the daily dose, and thus is present in a half or a quarter of any of the dosages recited above. In these embodiments, each dose fraction is taken separately over time to spread the dose across the day.

In some embodiments, the second component comprising a substituted or unsubstituted retinoid compound is administered at a dosage that it is sufficiently low to avoid toxicity, whilst still maintaining the required pharmaceutical effect. In some embodiments, the dosage of the second component comprising a substituted or unsubstituted retinoid compound depends on the bioavailability of the retinoid compound. In some embodiments, the bioavailability of the substituted or unsubstituted retinoid compound, varies depending upon whether it is a natural or synthetic product. In some embodiments, the bioavailability of the substituted or unsubstituted retinoid compound, may vary depending on whether it has been adapted to improve its bioavailability. In some embodiments, the second component comprising a substituted or unsubstituted retinoid compound is in a form suitable for providing a daily dosage of 250 mg or less, 200 mg or less, 150 mg or less, 100 mg or less, 50 mg or less, or 15 mg or less. In some embodiments, the second component comprising a substituted or unsubstituted retinoid compound is in a form suitable for providing a daily dosage of 10,000 μg or less, 5,000 μg or less, or 3,000 μg or less. In some embodiments, the second component comprising a substituted or unsubstituted retinoid compound is in a form suitable for providing a daily dosage of 50 μg or more, 100 μg or more, or 200 μg or more. In some embodiments, the second component comprising a substituted or unsubstituted retinoid compound is in a form suitable for providing a daily dosage from 200-15,000 μg, from 200-5000 μg, or from 200-3000 μg.

In some embodiments, daily dosage of the composition comprising a first component and a second component is provided in the form of one or more capsules or tablets. In some embodiments, daily dosage of the composition comprising a first component and a second component is provided in the form of 2 or 4 capsules or tablets. In these embodiments the capsules or tablets are taken during the course of a single day, such as one in the morning and one in the evening, or four spread evenly across the day, or two capsules or tablets simultaneously twice a day.

### Methods for Preparation

In some embodiments, are provided, methods for preparing the compositions described herein, suitable for use in treatment of rosacea. In some embodiments, any methods known in the art for blending or mixing various components of the composition are employed. In some embodiments, the methods employed are methods for blending and/or mixing powders. In some embodiments, the method comprises mixing a first component with one or more excipients and/or additives, optionally with a second component, and optionally a further component to form the composition. In some embodiments, the first, second and/or further components are each, separately from each other, mixed with one or more excipients and/or additives before being mixed together to form the composition. In some additional embodiments, the components, additives, and/or excipients are added sequentially to the mixture during the mixing process.

In some embodiments, the selection of the excipients and the method of blending are adapted in order to overcome any mixing, flow and fill issues or punch issues with the composition. In some embodiments, the composition comprising a first component comprising a substituted or unsubstituted DIM or a BR-DIM, is provided in micro-encapsulated form, such that the powder particles have a tendency to clump together. In some embodiments, the composition comprising a first component, comprising a substituted or unsubstituted DIM or a BR-DIM is blended using a method that is adapted to avoid creating hot spots of increased concentrations of the active ingredients. In some embodiments, the composition comprising a first component, optionally a second component, and optionally a further component, is blended using a method that involves short processing/blending times, to protect the composition from light and air, wherein the composition is hygroscopic and light sensitive. In some embodiments, the composition comprising a first component, optionally a second component, and optionally a further component, is prepared in the form of a powder, and the powder is protected from both light and air, during storage.

In some embodiments, one or more of microcrystalline cellulose, magnesium silicate, tricalcium phosphate, and magnesium stearate (a traditional lubricant) are employed as additives and excipients, in preparing the compositions described herein, to help with flow characteristics and/or lubrication. In some embodiments, other additives and excipients known in the art are employed if desired. In some embodiments, the composition comprises 50.0-65.0% by weight of tri-calcium phosphate. In some embodiments, the composition comprises, 55.0-60.0% by weight, or 57.0-59.0% by weight of tri-calcium phosphate. In some embodiments, the composition comprises about 58% by weight of tri-calcium phosphate. In some embodiments, the composition comprises about 58.3% by weight of tri-calcium phosphate.

In some embodiments, the correct blending of all ingredients is desirable in achieving uniform capsule fills of the compositions as described herein. In some embodiments, the correct blending of all ingredients is desirable in achieving uniform capsule fills of the compositions as described herein. In some embodiments, a V-blender is highly effective for successful mixing. In some embodiments, a minimum 316 grade stainless steel vessel is used for the mixing process. In some embodiments, sieving is performed at one or more of the start, the middle, and the end of the mixing process. In some embodiments, blend studies to confirm blend uniformity are completed to validate the method and formulation, using methods and techniques known in the field.

### Certain Definitions

The term “DIM” as used herein, refers to a substituted or unsubstituted diindolylmethane compound, of Formula 1.

The term “BR-DIM” or “BioResponse DIM” as used herein, refers to an unsubstituted DIM, provided by BioResponse LLC.

### Examples

The following specific, non-limiting examples are to be construed as merely illustrative, and do not limit the present disclosure of the scope of the disclosure. Without further elaboration, it is believed that one skilled in the art can, based on the description herein, utilize the present disclosure to its fullest extent.

### Example 1: Manufacture of a BR-DIM-Vitamin a Composition and Treatment of Rosacea Using the Composition

### Manufacture of Composition

A batch of BR-DIM formulation (Skin Accumax™) was manufactured. The details of manufacture and formulation are set out below.

### Formulation

1 capsule contains:
75 mg BR-DIM (BioResponse LLC)
200 μg vitamin A palmitate retinol equivalents (366.4 μg retinyl palmitate). This is equivalent to 2.666 mg of 250,000 IU/g vitamin A palmitate (BASF)
175 mg tri-calcium phosphate
20 mg microcrystalline cellulose
11.5 mg vitamin C (ascorbic acid)
10 mg fumed silicon dioxide (or a fine particle precipitated silica)
6 mg magnesium stearate

### Blending Method for Formulation

Mixing was carried out in a sequential process according to the following protocol:
1. The BR-DIM was sieved through a fine sieve and then mixed with the magnesium stearate, half of the tricalcium phosphate and the silicon dioxide for 15 mins in a V mixer. The mixed powder was transferred into a food grade bag.
2. Separately, the vitamin C and retinyl palmitate were mixed with the second half of the tricalcium phosphate and the microcrystalline cellulose for 15 mins in a V mixer.
3. The mixed powders obtained from 1 and 2 were blended together for 20 mins in a V mixer, then sieved, and mixed further for 1 hour and 45 minutes in a V mixer.
4. The blended powder was transferred to a suitable opaque and air-tight container to ensure minimal contact with light and oxygen.
5. A sample was taken from the top and bottom of the batch to test for uniformity of mixing.

### Encapsulation

The formulation referred to above in some embodiments is encapsulated. The BR-DIM is spray dried into particles of approximately 8 to 12 microns in diameter. BR-DIM is known to be light sensitive. Therefore, opaque capsules are preferred, typically, size “0” capsules. Light resistant containers are preferred.

### Patient Testing

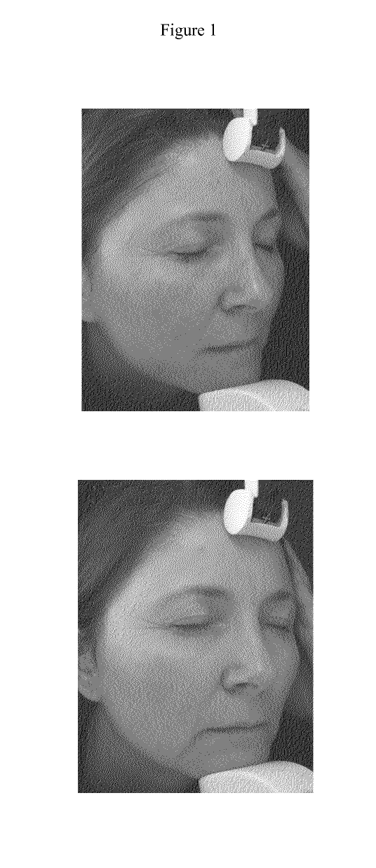

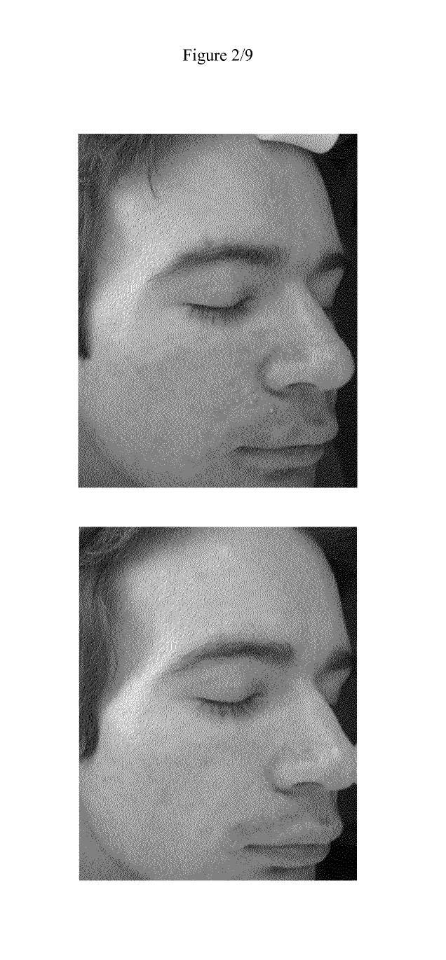

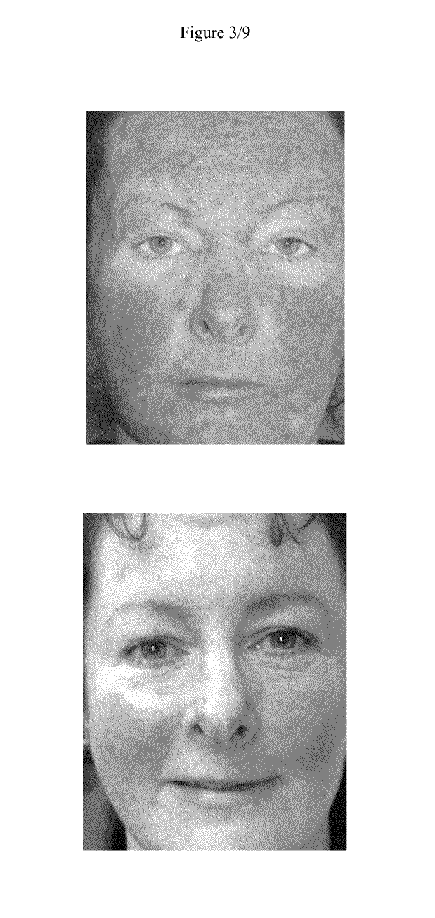

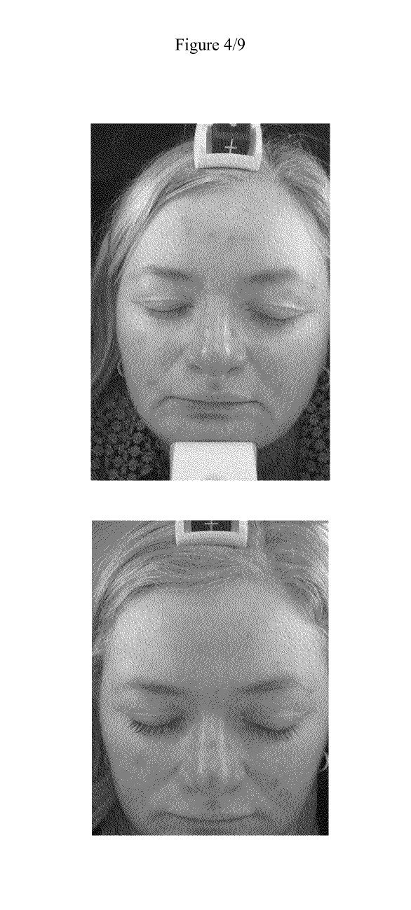

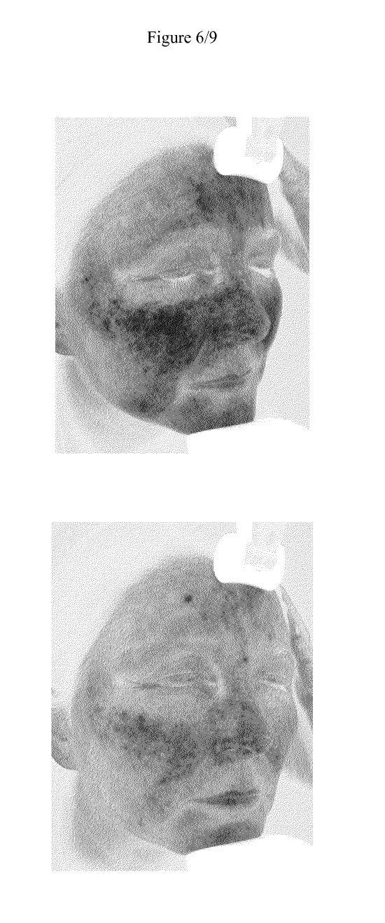

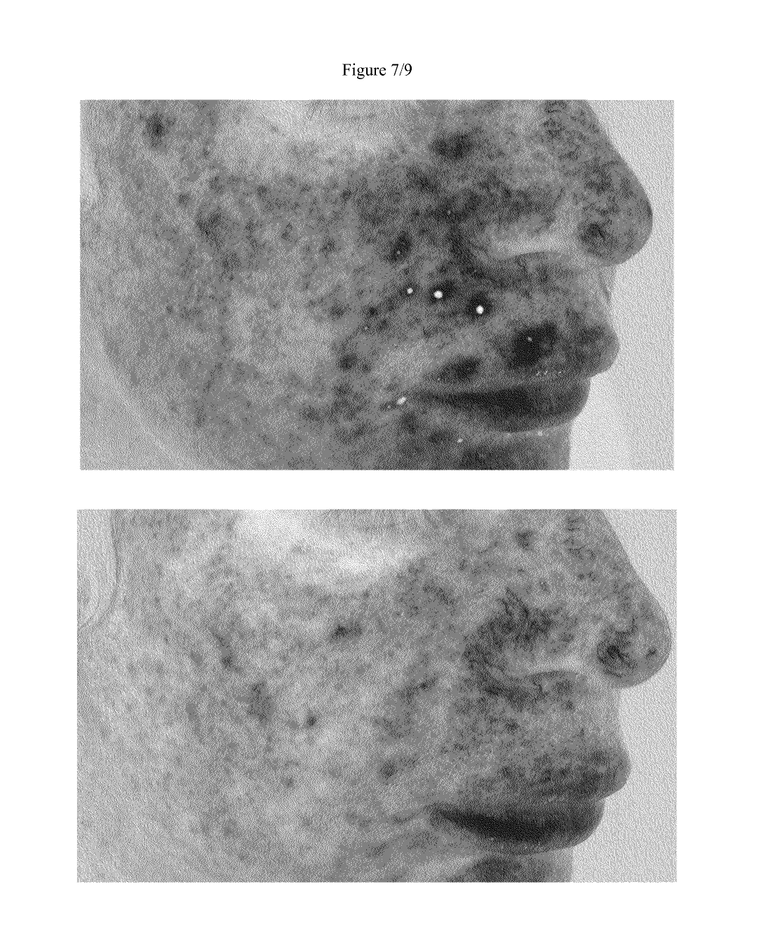

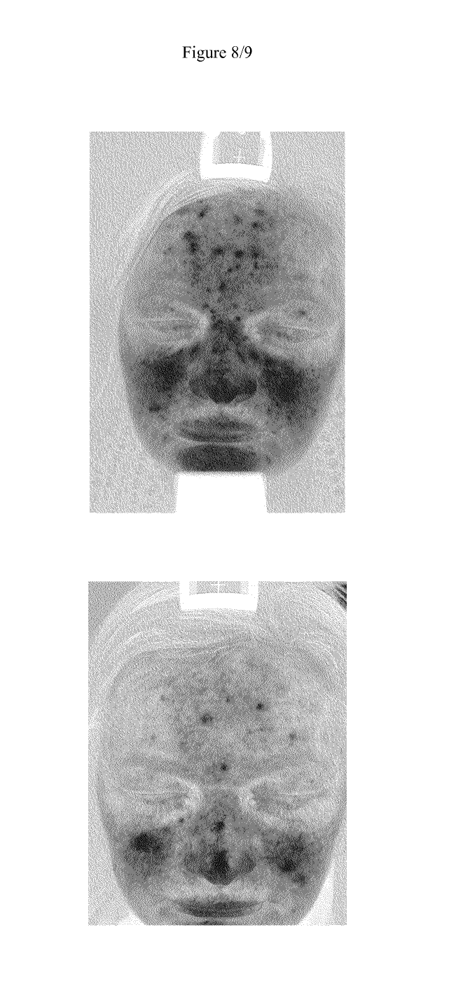

In the following, results are presented for five different patients suffering from rosacea, A1, A2, A3, B1, and B2. Three of the patients (A1, A2 and A3) were asked to take the formulation at home for several months in accordance with the dosage instructions provided with the packaged product on sale, and asked to return to provide feedback and analysis at the end of the trial. The remaining two patients (B1 and B2) were more closely monitored over a period of several months when taking the BR-DIM formulation, and asked to provide ongoing feedback. FIGS. 1, 2 and 3 show before and after photographs of patients A1, A2 and A3 respectively and FIGS. 4 & 5 show before and after photographs of patients B1 and B2. FIGS. 6, 7, 8 & 9 show before and after images using RBX™ VISIA Complexion Analysis technology (Canfield Imaging Systems, Fairfield, N.J., U.S.) of patients from each group (patients A1, A2, B1 and B2 respectively) to analyze vascular condition (redness).

### Patients A1, A2 and A3

Patients A1, A2 and A3 were provided with the formulation (Skin Accumax™) and asked to take 2 or 4 capsules per day for up to 6 months of treatment. Initial photographs and VISIA redness images were obtained as detailed above. After approximately 3 months the patients were recalled and further photographs and VISIA redness images were taken. The results are depicted in FIGS. 1, 2, 3, 6 and 7.

Patient A1 was asked to provide a verbal assessment of the result and stated “Skin Accumax has given me back my life, it is brilliant. Within two weeks I could see the visible redness diminish”. However, in order to provide a more detailed analysis of the results, the further more detailed tests on patients B1 and B2 were conducted.

### Patients B1 and B2

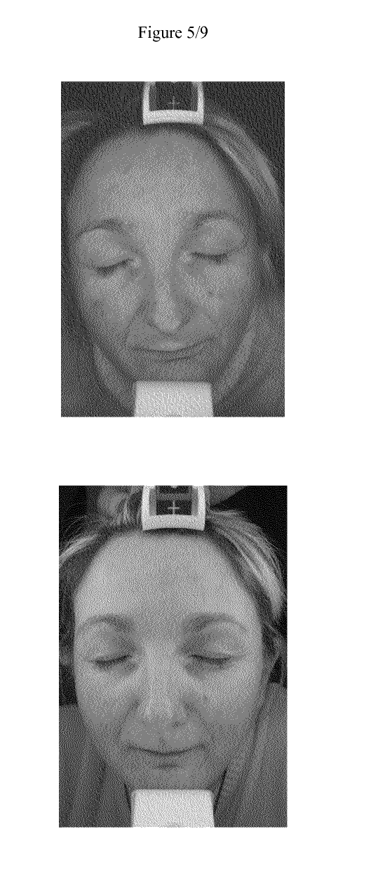

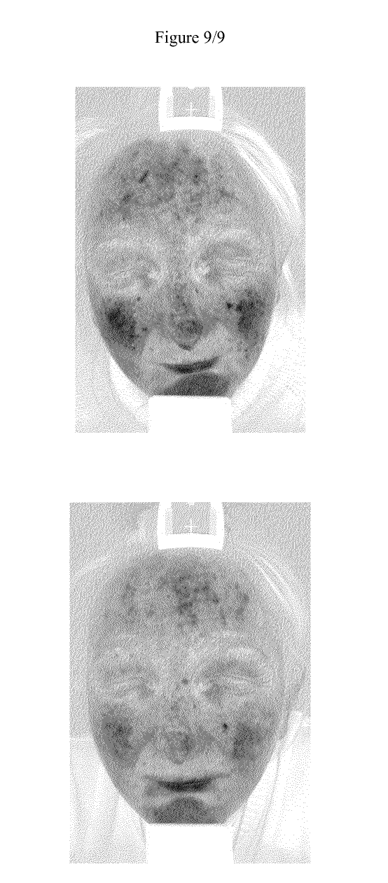

Patients B1 and B2 were provided with the formulation (Skin Accumax™) and asked to take 4 capsules per day for 6 months and 3 months respectively. Initial photographs and VISIA redness images were obtained as detailed above. They were also asked to self-report after each 1 month period and score 5 areas: redness, flushing, dryness, red bumps, and swelling of the skin, on a scale of 0-6, with 0 being the best. The self-reporting results are shown in Tables 1 and 2 for patients B1 and B2 respectively. Patient B1 stopped self-reporting after 5 months. After 6 months and 3 months respectively, the patients were recalled and further photographs and VISIA redness images taken. The results are depicted in FIGS. 4, 5, 8 and 9.

TABLE 1
self reporting results for patient B1

| | Redness | Flushing | Dryness | Red bumps | Swelling |
|---|---|---|---|---|---|
| Month 1 | 4 | 4 | 3 | 3 | 6 |
| Month 2 | 3 | 3 | 3 | 3 | 5 |
| Month 3 | 3 | 3 | 2 | 2 | 4 |
| Month 4 | 2 | 3 | 2 | 3 | 3 |
| Month 5 | 1 | 3 | 1 | 2 | 1 |

TABLE 2
self reporting results for patient B2

| | Redness | Flushing | Dryness | Red bumps | Swelling |
|---|---|---|---|---|---|
| Month 1 | 6 | 6 | 5 | 3 | 1 |
| Month 2 | 2 | 4 | 5 | 3 | 0 |
| Month 3 | 1 | 2 | 4 | 1 | 0 |

These results demonstrated the significant potential of the exemplary formulation (Skin Accumax™), combining BR-DIM and vitamin A, in the treatment of rosacea.

While specific embodiments described herein have been shown and described in detail to illustrate the application of the principles of the disclosure, it will be understood that the disclosure may be embodied otherwise without departing from such principles. Numerous variations, changes, and substitutions will be understood by those skilled in the art without departing from the disclosure. It should be understood that various alternatives to the specific embodiments of the disclosure described herein may be employed in practicing the disclosure. It is intended that the claims define the scope of the disclosure, and that methods and structures within the scope of these claims and their equivalents be covered thereby.

1. A composition for use in treating rosacea, the composition comprising:
(a) a first component comprising a substituted or unsubstituted diindolylmethane; and
(b) optionally, a second component comprising a substituted or unsubstituted retinoid compound.

2. The composition according to claim 1, wherein the composition for use in treating rosacea further comprises a further component selected from one or more of, a substituted or unsubstituted retinoid compound, an antibiotic compound, a substituted or unsubstituted azelaic acid compound, an oral contraceptive compound, sulphur, a sulphur-containing compound, a substituted or unsubstituted salicylic acid compound, a substituted or unsubstituted resorcinol compound, a plant product, a mineral, a vitamin, and a neutraceutical product.

3. The composition according to claim 1 or claim 2, wherein the first component is a diindolylmethane of Formula 1: 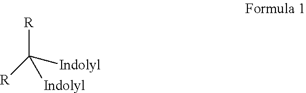
wherein the R groups are independently selected from hydrogen atoms and C₁-C₆ hydrocarbon substituents;
and wherein the indolyl groups are independently selected from indole-3-yl and indole-2-yl groups;
and wherein the indolyl groups are unsubstituted, or are substituted with one or more C₁-C₆ hydrocarbon substituents.

4. A composition according to claim 3 wherein the first component is selected from a group consisting of a substituted or unsubstituted 3,3′diindolylmethane, of Formula 2, and a substituted or unsubstituted 2,2′diindolylmethane, of Formula 3: 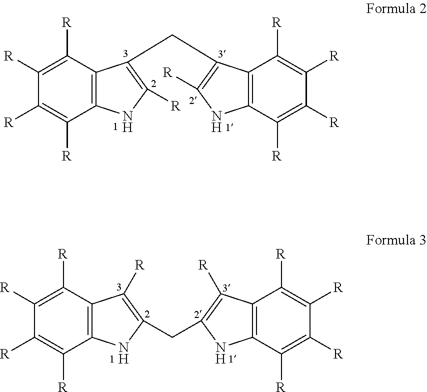
wherein the R groups are independently selected from hydrogen atoms, and C₁-C₆ hydrocarbon substituents.

5. The composition according to claim 4 wherein the first component is a 3,3′diindolylmethane, of Formula 4 or a 2,2′diindolylmethane, of Formula 5:

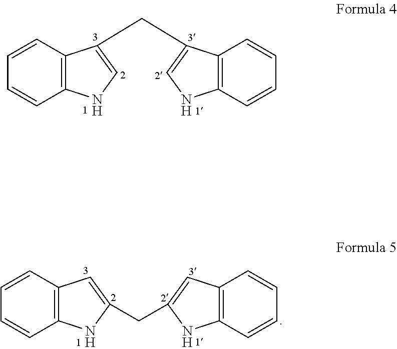 [SMILES: c1ccc2c(Cc3c[nH]c4ccccc34)c[nH]c2c1 ; c1ccc2[nH]c(Cc3cc4ccccc4[nH]3)cc2c1]

6. The composition according to any preceding claim, wherein the substituted or unsubstituted retinoid compound is selected from a substituted or unsubstituted first generation retinoid, a substituted or unsubstituted second generation retinoid, and a substituted or unsubstituted third generation retinoid.

7. The composition according to claim 6, wherein the substituted or unsubstituted first generation retinoid is selected from a substituted or unsubstituted retinol, a substituted or unsubstituted retinal, a substituted or unsubstituted tretinoin, a substituted or unsubstituted isoretinoin, and a substituted or unsubstituted alitretinoin, wherein the substituted or unsubstituted tretinoin is selected from retinoic acid and Retin A.

8. The composition according to claim 6 or claim 7, wherein the first generation retinoid is vitamin A.

9. The composition according to claim 6, wherein the second generation retinoid is selected from a substituted or unsubstituted etretinate, and a substituted or unsubstituted acitretin.

10. The composition according to claim 6, wherein the third generation retinoid is selected from a substituted or unsubstituted tazarotene, a substituted or unsubstituted bexarotene, and a substituted or unsubstituted adapalene.

11. The composition according to any of claims 2-10, wherein the antibiotic compound is selected from tetracycline, oxytetracycline, minocycline, doxycycline, erythromycin, and trimethoprim.

12. The composition according to any of claims 2-11, wherein the azelaic acid compound comprises an antibacterial property, an anti-inflammatory property, or a combination thereof.

13. The composition according to any of claims 2-12, wherein the oral contraceptive comprises a female hormone, a compound with female hormone activity, a compound with female hormone activity capable of counteracting the effect of a male hormone, or a combination thereof.

14. The composition according to claim 13, wherein the female hormone is oestrogen and the male hormone is testosterone.

15. The composition according to any of claims 2-14, wherein the resorcinol compound comprises an antibacterial property, an anti-inflammatory property, or a combination thereof.

16. The composition according to any of claims 3-15, wherein an unsubstituted compound comprises only hydrogen atom substituents, and wherein a substituted compound comprises one or more C₁-C₆ hydrocarbon substituents.

17. The composition according to any preceding claim, wherein the type of rosacea is selected from Pustular rosacea, Papulopustular rosacea, Erythemato-telangiectatic rosacea, Granulomatous rosacea, Rosacea conglobata, Rosacea fulminans, and Phymatous rosacea.

18. The composition according to any preceding claim, wherein the composition is suitable for oral or topical administration.

19. The composition according to claim 18, wherein the composition is in the form of a tablet, capsule, gel, cream or ointment.

20. A pharmaceutical kit comprising a first component and optionally a second component, for use in treating a skin condition, by separate, simultaneous or sequential administration of the first and the second component, wherein the first and second components are as defined in any of claims 1-19.

21. The composition or kit according to any preceding claim, wherein the substituted or unsubstituted diindolylmethane is synthetic, or is a natural product obtained from a Brassica plant.

22. The composition or kit according to claim 20 or claim 21, wherein the substituted or unsubstituted diindolylmethane is adapted for increased bioavailability.

23. The composition or kit according to claim 22, wherein the substituted or unsubstituted diindolylmethane is BR-DIM.

24. The composition or kit according to any preceding claim comprising, the first component, the second component, the further component, an excipient or adjuvant, and a combination thereof.

25. The composition or kit according to claim 24, wherein the first component further comprises one or more lipophilic compounds selected from vitamin E and phosphatidylcholine.

26. The composition or kit according to any preceding claim, wherein the first component is in a form suitable for providing a daily substituted or unsubstituted diindolylmethane dosage of up to 1500 mg or less.

27. The composition or kit according to claim 26, wherein the first component is in a form suitable for providing a daily substituted or unsubstituted diindolylmethane dosage of about 10 mg to about 750 mg.

28. The composition or kit according to any preceding claim, wherein the first component is for treating rosacea, and is in a form suitable for providing a daily substituted or unsubstituted diindolylmethane dosage of about 150 mg to about 650 mg, about 250 mg to about 550 mg, about 300 mg to about 500 mg, about 400 mg to about 450 mg, or about 500 mg.

29. The composition or kit according to any of claims 26-28, wherein the first component is present in a dose which is a fraction of the daily dose, preferably a half of the daily dose, or a quarter of the daily dose.

30. The composition or kit according to any preceding claim, wherein the second component is in a form suitable for providing a daily retinoid compound dosage of about 200 to about 3000 μg.

31. The composition or kit according to any preceding claim, wherein the daily dosage is provided in the form of one or more capsules or tablets.

32. A method for treating rosacea, comprising administering a composition or components of a kit, as defined in any preceding claim, to a subject in need thereof.

33. A method for preparing a composition as defined in any of claims 1-19, comprising mixing the first component with one or more of the second component, the excipients, and the additives, to form the composition.

34. The method according to claim 33, wherein the first and second components are each, separately from each other, mixed with one or more excipients, additives, or a combination thereof, before being mixed together to form the composition.

35. A substituted or unsubstituted diindolylmethane for use in treating rosacea.
